(19) United States
(12) Patent Application Publication
(10) Pub. No.: US 2009/0292328 A1
(43) Pub. Date: Nov. 26, 2009

(54) Medical Device

(76) Inventors: Corlius Fourie Birkill, Rietondale (ZA); Roche Janse Van Rensburg, Garsfontein (ZA)

Correspondence Address: OSTROLENK FABER GERB & SOFFEN, 1180 AVENUE OF THE AMERICAS, NEW YORK, NY, 100368403, US

(21) Appl. No.: 12/085,657

(22) PCT Filed: Nov. 24, 2006
(86) PCT No.: PCT/IB2006/003345

§ 371 Date: May 29, 2008

(30) Foreign Application Priority Data
Nov. 30, 2005 (ZA) 2005/09686

(51) Int. Cl. A61N 1/36; A61N 1/30

(52) U.S. Cl. 607/3; 607/152; 607/149; 607/66

(57) ABSTRACT

A disposable medical device includes an electrode pad adapted to be placed in contact with a human or animal body and an electrical signal generator permanently attached to and in close proximity with the electrode pad and operable to generate an electrical signal, the signal generator having two output terminals, at least one of which is electrically operably connected to the electrode pad.

### FIELD OF THE INVENTION

This invention relates to a medical device. More particularly, the invention relates to a disposable medical device incorporating an electrode pad.

### BACKGROUND TO THE INVENTION

Many forms of electronic devices for the treatment or diagnosis of medical conditions in humans or animals are known. Many of these devices require the application of electrodes to various parts of the human or animal body. These electrodes are generally provided separately from the main device in sterile packs and are applied to the relevant portions of the patient's body at the time of treatment either by means of an adhesive or a gel which improves conductivity. The device itself is often cumbersome and is generally not disposable and is connected to the electrodes by means of wires and connection terminals.

One such device is a neurostimulating device used for the locating of specific nerves and the treatment of these nerves with anesthetics or other drugs. Such neurostimulating devices involve the use of an electrode which is placed on the patient's body and which is operably connected to a signal generator by means of a wire. A probe and/or a hypodermic drug dispenser, including a hypodermic needle, are attached to a terminal of the signal generator. By generating an electrical signal and applying it across the electrode and the probe and/or hypodermic needle, it is possible to both precisely locate the position of a particular nerve and to apply a drug or anesthetic by means of the hypodermic needle on or proximate the nerve. The problem with present transdermal neurostimulators and delivery systems is that they are difficult to manipulate single-handedly. The operator must simultaneously manipulate the signal generator and the probe and/or hypodermic needle. It would therefore be an advantage to have the signal generator, incorporating manipulative dials and read outs relating to the strength and form of the signal generated thereby, in close proximity to the part of the human body on which the probe and/or hypodermic needle is being used. It would be a further advantage for the entire combination of equipment to be provided in a sterile packaging and to be disposable after use.

A further use for a medical device including a potential difference (voltage) generator is to be found in a drug delivery system for the delivery of drugs by means of iontopheresis. In such a delivery system, a charged ionic drug is placed on the skin of a patient and an electric charge of the same polarity as that of the drug is applied to the region of the skin. This allows a direct current to drive the drug into the skin. Iontopheresis-aided delivery may take advantage of sweat ducts, sebaceous glands, hair follicles and imperfections in the skin to achieve penetration. Alternatively, applying an electrical potential across a portion of the skin may alter its permeability, possibly creating potential-dependant pores in lipid bilayer membranes. The use of such devices is appropriate in the delivery of local anesthetic prior to skin puncture or painful dermal procedures, the treatment of excessive sweating of the hands and feet (i.e. palmoplantar hyperhydrosis), local drug delivery for agents such as non-steroidal anti-inflammatory drugs (NSAID's) or corticosteroids for musculoskeletal inflammatory disorders, and other applications. Again, it would be an advantage to provide a device in which the electrical potential could be controlled locally at the site of application by means of a signal generator. It would further be an advantage for an electrode pad, signal generator and, possibly, a topically applied drug to be prepackaged in a sterile package and to be disposable after use.

### OBJECT OF THE INVENTION

It is an object of this invention, at least in part, to achieve the advantages set out above and to overcome the problems alluded to.

### SUMMARY OF THE INVENTION

A disposable medical device including
an electrode pad adapted to be placed in contact with a human or animal body; and
an electrical signal generator permanently attached to and in close proximity with the electrode pad and operable to generate an electrical signal, the signal generator having two output terminals, at least one of which is electrically operably connected to the electrode pad.

The electrode pad may have an adhesive on its contact surface and a removable film covering the contact surface and adhesive, the film being removable prior to use. Preferably, the electrode pad and signal generator are in abutment when in use. In one embodiment of the invention, the electrode pad and signal generator comprise an integral one-piece unit. In another embodiment of the invention, the electrode pad and the signal generator have complementary connectors for connecting the electrode pad and signal generator directly to one another without the aid of an electrical lead.

The signal generator may be intended for single use and disposal thereafter and may be battery powered, the battery being installed at the point of manufacture.

The signal generator may be activated for single use by means of a pull tag, having the effect of completing the power circuit and thereby enabling the device.

The signal generator may be operable to generate a range of signals of various selectable wave forms and amplitudes.

Further, the signal generator may include
means for selecting the signal wave form and amplitude or signal current; and
a digital readout for reading the wave form and/or amplitude of the signal or the current.

The means for selecting the signal wave form and amplitude or signal current may be a dial or switch.

The signal generator may be operable to generate a monophasic square wave pulse of selectable amplitude.

In a preferred embodiment of the invention, the medical device comprises a transdermal neurostimulator, a first of the terminals of the signal generator being applied to the electrode pad and comprising an anode for the device and a second terminal of the signal generator being attached to a connector for connection to a transdermal probe and/or a drug delivery device including a hypodermic syringe and needle and comprising the cathode of the device.

In another embodiment of the invention, the electrode pad comprises two electrically insulated regions, a first of which provides a cathode and a second of which provides an anode, the first terminal of the signal generator being in electrical contact with the first region of the electrode pad, and the second terminal of the signal generator being in electrical contact with the second region thereof. Then, the signal generator may be operable to selectively vary the signal strength between the anode and cathode.

One of the regions of the electrode pad may be impregnated with an ionic drug of choice, drug delivery being made by means of iontopheresis and the rate of delivery of the drug being varied by selected variation of the signal strength between the regions of the electrode. Instead, an ionic drug may be laced within an electrode gel which is applied to the electrode at the time of application, drug delivery being made by means of iontopheresis and the rate of delivery of the drug being be varied by selected variation of the signal strength between the regions of the electrode.

According to a further aspect of the invention there is provided a kit for a disposable medical device, the kit including
an electrode pad adapted to be placed in contact with a human or animal body; and
an electrical. signal generator permanently attached to and in close proximity with the electrode pad and operable to generate an electrical signal, the signal generator having two output terminals, at least one of which is electrically operably connected to the electrode pad;
the electrode pad and the electrical signal generator being provided in sterile wrapping and disposable after single use.

Thus, in one embodiment of the invention, in use, the entire miniaturized signal generator and electrode pad are removed from sterile packaging and applied, using either a self adhesive layer carried on the contact surface of the electrode pad or by means of an electrode gel, to the body of the patient at a location proximate the area of treatment. The hypodermic applicator is connected to the second terminal of the signal generator by means of a cable. The operator selects a suitable amplitude or current strength and signal wave form for treatment of the nerve in question and, by means of the hypodermic needle, locates the nerve. Since the device is proximate the area of treatment, it is simple for the operator to both read the amplitude and form of the signal in use and to alter these, as appropriate, while continuing to manipulate the hypodermic needle. A reaction of the patient to the applied stimulus determines that the hypodermic needle is proximate the relevant nerve, and an anesthetic or other drug may be locally applied to the nerve in question. After treatment, the entire electrode pad signal generator combination may be discarded.

### BRIEF DESCRIPTION OF THE DRAWINGS

The invention is now described, by way of example only, with reference to the accompanying diagrammatic drawings. In the drawings:

FIG. 1 shows a schematic view of a first embodiment of a disposable medical device, in accordance with the invention;

FIG. 2 shows the device of FIG. 1 in place for treatment on the human body; and

FIG. 3 shows a second embodiment of the disposable medical device in accordance with the invention.

### DETAILED DESCRIPTION OF THE DRAWINGS

In the drawings, reference numeral 10 generally refers to a disposable medical device in accordance with the invention.

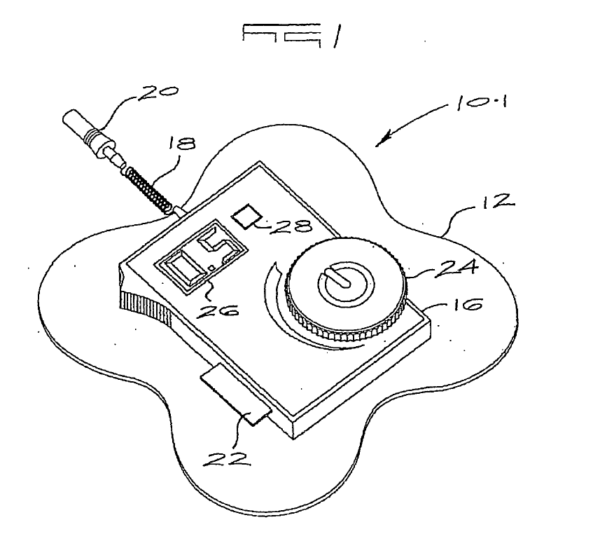

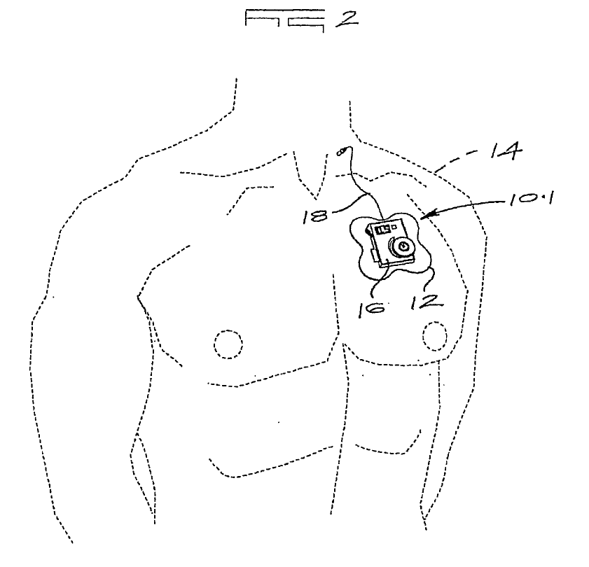

In FIG. 1, the device 10 comprises a transdermal neurostimulating device 10.1. The device comprises an electrode pad 12, which is adapted to be placed in contact with the human body 14 at a location proximate to an area of treatment for a selected nerve (as shown in FIG. 2). Attached to the electrode 12 is an electrical signal generator 16, which is operable to generate an electrical signal between two terminals (not shown). One of these terminals is electrically connected to the electrode pad 12 so that effectively this terminal is in contact with the human body at the point of application of the pad 12. A second terminal of the signal generator 16 terminates in an electrical cable 18, a free end of which contains a connector 20 for connection to a drug delivery system comprising a hypodermic syringe and needle (not shown). The electrode pad 12 provides an anode for the signal generator 16, while the needle provides a cathode for the signal generator 16. The needle is in electrical contact with the second terminal of the signal generator 16. The signal generator 16 is designed for single use and is battery powered, the battery (not shown) being installed at the point of manufacture. In order to activate the device, a pull tag 22 is provided which, on operation, closes a circuit between the battery and the signal generator circuit. The signal generator 16 is operable to generate a number of preselected wave forms, the amplitude and shape of each of which may be manipulated by means of a dial 24. In the embodiment 10.1, the wave forms are monophasic square wave pulses. A liquid crystal readout 26 is provided on the signal generator 16, the readout 26 showing the amplitude of the applied signal at any time. An indicator 28 is provided to indicate by means of lights the range and/or wave form selected.

The neurostimulator device 10.1 is provided in a sterile condition in a sterile wrapper (not shown). In use, the neurostimulator device 10.1 is removed from the sterile wrapper, applied to the body 14 of the person to be treated in a location proximate the area of treatment and the electrode pad 12 is adhered to the body 14 by means of a self adhesive applied to a contact surface of the pad 12 at the point of manufacture or a typical contact gel. The neurostimulator device 10.1 is activated by pulling the pull tag 22 and the hypodermic probe and/or needle are connected to the connector 20 of the cable 18. The operator may then manipulate the hypodermic needle and, at the same time, observe the readout 26 and manipulate the signal strength without difficulty. Once it is apparent that the hypodermic needle is proximate the nerve to be treated, the contents of the hypodermic syringe, being typically an anesthetic or other treatment drug, may be locally applied very precisely to the nerve in question.

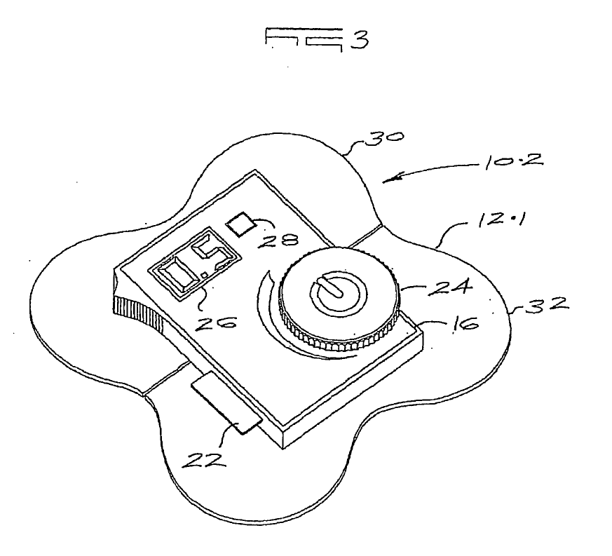

In FIG. 3, an embodiment 10.2 of the device 10 comprising a drug delivery system is shown. In this embodiment 10.2, the electrode pad 12.1 comprises two separate electrically insulated regions 30,32, a first of which 30 comprises an anode and a second of which 32 comprises a cathode. The signal generator terminals are connected one to each of the regions of the electrode pad 12.1. Thus, a potential difference is set up in the human body between two regions in contact with the regions 30,32 of the electrode pad 12.1. In this case, an ionic drug for application by means of iontopheresis may be carried in an electrode gel which is applied to the region 30,32 of the electrode pad 12.1 having the same polarity as the drug. Instead, the drug may be provided at the point of manufacture, already applied to the contact surface of the electrode 12.1. The rate of delivery of the drug may be controlled by manipulation of the potential difference between the regions 30,32 of the electrode pad 12.1. Thus, from time to time, the samples may be taken from the patient to determine whether the correct strength of drug has been delivered and the signal generator 16.1 may be controlled to either increase or decrease the rate of delivery of the drug to the patient.

By means of the invention, there is provided a simple and effective means of providing a controlled electrical field in the human body which may be precisely located and controlled. The entire device, including signal generator and electrode pad, may conveniently be used for a single application and discarded. Advantageously, a combination of the signal generator and electrode pad may be provided in a sterile packaging. The proximity of the controls and read out of the signal generator to the area of topical treatment provides an advantage over existing treatment devices.

1-18. (canceled)

19. A disposable medical device comprising:
an electrode pad operable for placement in contact with a human or animal body;
an electrical signal generator permanently attached to and in close proximity with the electrode pad and operable to generate an electrical signal, the signal generator having two output terminals, at least one of which is electrically operably connected to the electrode pad; and
a transdermal neurostimulator, wherein a first output terminal of the signal generator is applied to the electrode pad which serves as an anode for the device and a second output terminal of the signal generator is attached to a connector for connection to the transdermal neurostimulator, the transdermal neurostimulator including a drug delivery device including a hypodermic syringe and needle that serve as a cathode of the device; and
wherein the electrode pad, the electrical signal generator and the transdermal neurostimulator are provided in sterile wrapping and disposable after single use.

20. The disposable medical device of claim 19, wherein the electrode pad further comprises:
an adhesive on its contact surface; and
a removable film covering the contact surface and adhesive, the film being removable prior to use.

21. The disposable medical device of claim 19, wherein the electrode pad and signal generator are in abutment when in use.

22. The disposable medical device of claim 21, wherein the electrode pad and signal generator are formed as an integral one-piece unit.

23. The disposable medical device of claim 21, wherein the electrode pad and the signal generator have complementary connectors for connecting the electrode pad and signal generator directly to one another without the aid of an electrical lead.

24. The disposable medical device of claim 23, wherein the signal generator is battery powered.

25. The disposable medical device of claim 24, wherein the battery is installed at a point of manufacture of the device.

26. The disposable medical device of claim 24, wherein the signal generator is activated for single use by means of a pull tag, having the effect of completing a power circuit and thereby enabling the device.

27. The disposable medical device of claim 19, wherein the signal generator is operable to generate a range of signals of various selectable wave forms and amplitudes.

28. The disposable medical device of claim 26, wherein the signal generator further comprises:
means for selecting at least one of the signal wave form, amplitude and signal current; and
a digital readout for reading the wave form, amplitude and current of the signal.

29. The disposable medical device of claim 27, wherein the signal generator is operable to generate a monophasic square wave pulse of selectable amplitude.

30. The disposable medical device of claim 19, wherein the signal generator is operable to selectively vary the signal strength between the anode and cathode.

31. A kit for a disposable medical device, the kit comprising:
an electrode pad operable for placement in contact with a human or animal body;
an electrical signal generator permanently attached to and in close proximity with the electrode pad and operable to generate an electrical signal, the signal generator having two output terminals, at least one of which is electrically operably connected to the electrode pad; and
a transdermal neurostimulator, wherein a first output terminal of the signal generator is applied to the electrode pad which serves as an anode for the device and a second output terminal of the signal generator is attached to a connector for connection to the transdermal neurostimulator, the transdermal neurostimulator including a drug delivery device including a hypodermic syringe and needle that serve as a cathode of the device; and
wherein the electrode pad, the electrical signal generator and the transdermal neurostimulator are provided in sterile wrapping and disposable after single use.
